(19) United States
(12) Patent Application Publication
(10) Pub. No.: US 2004/0101153 A1
(43) Pub. Date: May 27, 2004

(54) Gas flow sensor, speaker system and microphone, utilizing measurement absolute of time-variations in absolute pressure

(76) Inventors: Oleg Grudin, Montreal Quebec (CA); Gennadiy Frolov, Montreal Quebec (CA); Leslie M. Landsberger, Westmount Quebec (CA)

Correspondence Address: OGILVY RENAULT, 1981 MCGILL COLLEGE AVENUE, SUITE 1600, MONTREAL, QC, H3A2Y3, CA

(21) Appl. No.: 10/275,255

(22) PCT Filed: May 8, 2001
(86) PCT No.: PCT/CA01/00647

(51) Int. Cl. A61F 11/06; H04R 1/20; H04R 17/02; H04R 19/04

(52) U.S. Cl. 381/357; 381/351; 381/354; 381/71.1

(57) ABSTRACT

This invention addresses a fundamental weakness in currently common microphones, and their applications in feedback for audio speaker systems. This limitation stems from the fundamental difficulties of typical membrane-type microphones in the frequency range 1-100 Hz. The self-noise of membrane-type microphones increases in this range approximately as 1/f, and membrane-type microphones are sensitive to parasitic inertial vibrations, which are usually very important in this frequency range. The removal of these limitations enables the use of such a sensor (APV sensor) for effective feedback in audio speakers, dramatically improving performance in the low-frequency range of 10-100 Hz. Without feedback, typical audio speakers suffer severe attenuation and/or distortion in this range.

### FIELD OF THE INVENTION

The present invention relates to sensors measuring time-variations in absolute pressure of gases at a point in space, and to sound reproduction and recording systems based on these sensors. The applications include the use of these sensors in systems of motional feedback (MFB) for improvement of audio speakers, and the use of these sensors in hybrid microphones. This invention concerns particularly the low-frequency end of the audio spectrum, as well as part of the infrasonic range, in order to improve microphone performance in this frequency range, and also to enable effective motional feedback for this range, for audio speaker systems.

### BACKGROUND OF THE INVENTION

The field of acoustic microphones is a mature field, having existed for several decades, with extensive variety of models commercially available, covering the frequency range from hundreds of kHz (ultrasonic) to Hz or fractions of Hz (infrasonic). In particular, in audio applications, the range from 20 Hz to 20 kHz is typically defined as the range which contains signal components of interest for human listening.

Membrane-type microphones typically have limited sensitivity and increased noise (having typically a 1/f dependence) at lower frequency. In addition to this, they are fundamentally prone to parasitic inertial vibrations, in the same frequency range as the low-frequency sound (pressure) signals of interest.

The concept of a flow-based low-frequency microphone is known from the article by Robert Fehr, “Infrasonic Thermistor Microphone,” Journal of the Audio Engineering Society, April 1970, Volume 18, No.2, pp.128-132. The microphone device consists of a thermistor arrangement, providing a thermoanemometer-type flow sensor, placed in the tube of a Helmholtz resonator, connecting a gas-volume chamber to the ambient. This flow-sensor measures flow in and out of the chamber through the tube, as a result of time-variations in ambient pressure at the inlet of the tube. This device operates at frequencies as low as 0.1 Hz, and is less sensitive to vibration than membrane-type microphones. On the other hand, the described device has certain limitations which prevent its use in practical low frequency audio applications. First, the described device uses discrete elements (thermistors) as the temperature-sensing elements of a thermoanemometer, which prevents achieving an arbitrarily low thermal inertia, resulting in a transition-frequency in the order of 1.5 Hz, above which signals are progressively attenuated, which in turn limits the available frequency range of the microphone. Second, the placement of these discrete elements (described as 330 μm in diameter in the article) within a flow channel makes the use of arbitrarily narrow flow channels difficult, thus limiting the available range of flow impedance.

Therefore, if the object is to detect sound in the frequency range down to 1 Hz or lower, the gas-volume chamber must have a substantial volume, for example hundreds of milliliters. For applications such as an audio microphone, or a sensor for use in motional feedback (MFB—see below), such large volumes are impractical. Third, the pressure-sensitivity (resolution) of the device described in the Fehr article is estimated at 1-10 Pa, while for audio applications sensitivity in the mPa range or better is required.

The MFB technique is well-known as an effective method to improve low-frequency fidelity of audio speakers by extending their frequency response in the lower frequency range down to approximately 20 Hz, and reducing low-frequency signal distortions. In general, the MFB method is based on a transducer detecting sound pressure (or a correlate thereof), generated by the speaker membrane, and negative feedback of the detected signal to a power amplifier. Comparison of the external electrical input signal with the measured sound pressure or with the speaker membrane motion, or other correlate, allows automatic adjustment of the delivered electric power, to compensate for distortions caused by certain complex nonlinear electro-mechanical properties of the speaker system.

Prior to the advent of mass-market digital sound reproduction equipment in the early 1980's with the compact disc (CD), audiophile sound reproduction was an expensive challenge and was foreign to most consumers. Today, any inexpensive CD player will reproduce an audiophile quality signal of the original recording. This signal is prone to being distorted by speaker systems for the low frequency component below, and around, say, 100 Hz, particularly when the amplitude of the low frequency component is great. Signal clipping may be clearly audible, and listeners may typically reduce the sound volume to reduce the effect of the distortion. Audiophiles still strive to achieve quality sound reproduction by using sophisticated speaker systems having a more faithful reproduction in the low frequency range. With effective MFB implemented, conventional mass-market consumer-quality level sound reproduction equipment, namely stereo amplifier and speakers, would be able to deliver audiophile (high-quality) reproduction. This means that relatively inexpensive, home-stereo equipment could approach competing with audio systems having substantially higher price, as long as MFB can be successfully implemented.

The primary limitation preventing MFB from being successfully and widely implemented today is the lack of a suitable (as evaluated by a combination of price, size, and performance characteristics), low-frequency microphone (i.e. sensor or pickup). A secondary limitation is the requirement for an independent power supply at the speaker, to implement MFB. A further limitation is that the speaker to be used with MFB requires special adaptation.

Motional feedback has generally been done in one of two ways: (1) using an accelerometer to measure speaker membrane acceleration (e.g. U.S. Pat. No. 4,573,189); and (2) using a microphone to measure sound pressure (e.g. U.S. Pat. No. 4,592,088).

The first method to accomplish MFB is based on the use of an accelerometer attached to the speaker membrane, and the detection of its acceleration instead of actual sound pressure. Complex technical problems must be solved to realize accelerometer-based MFB. Very high technical performance is required from the accelerometer, for two reasons. Acceleration of the speaker membrane equals a=(2πf)²x, where f is frequency and x is amplitude of the membrane oscillations. The upper limit of acceleration aₘₐₓ≈100 g can be estimated at the high end of the frequency range, say 120 Hz, and maximum motional amplitude of the speaker membrane of 2 mm. Both values are typical for audio speakers. MFB should be also effective at lower frequencies down to approximately several Hz (to provide effective feedback at frequencies higher than 20-25 Hz), and loudness of four orders of magnitude lower than its maximum value, due to human physiological properties and the range of human hearing. Thus, the minimum required detectable acceleration can be estimated at a level of milli-g or lower. Therefore a high-performance accelerometer is needed, having linear dynamic range of at least five orders of magnitude, and capable of measuring high acceleration up to 100 g.

It has also been found (U.S. Pat. No. 4,727,584) that an accelerometer attached to the membrane of an audio speaker responds to undesirable parasitic signals due to occasional low frequency (e.g. several Hz) oscillations and high frequency (several hundred Hz) instabilities. These effects are caused by air pressure variations inside the speaker enclosure and acoustic resonances of sound waves interacting with the accelerometer. These factors, which are difficult to control in manufacturing, result in non-trivial design constraints for the speaker driver, including: (1) employing a speaker membrane having a “trumpet” shape appearance. (2) inverting the dust cups, (3) the placing of weights on the loudspeaker coil in selected locations circumferentially with respect to the accelerometer to minimize instability effects.

As a result, the use of accelerometer-based MFB requires not only an excellent, high performance accelerometer, but also a custom-designed speaker driver. Attempts to attach a motion-sensitive element to ordinary commercially-available speaker drivers tend to give marginal results.

The second way of obtaining MFB is based on direct measurements of sound pressure which are currently accomplished by a microphone located in the vicinity of the speaker membrane. Due to the dynamics of poles and phase shifts in a feedback configuration, if bass improvement in the range 20-150 Hz is desired, the microphone needs to operate at frequencies from several Hz to several hundred Hz. Effective motional feedback, able to suppress distortions at frequencies in the range of 20-25 Hz, would require reliable operation of a microphone with high signal-to-noise ratio (SNR) down to 1-3 Hz. Existing microphones have inherent increase of noise at low frequencies, and substantial decrease of sensitivity at low frequencies. That is why even the best available microphones have low cut-off frequency at approximately 20 Hz.

In general, a microphone can measure acoustic pressure inside or outside the speaker enclosure. Inside the enclosure, measurable pressures may reach 180-200 dB SPL (sound pressure level), which is much higher than the typical upper limit of a microphone's operating range (120-130 dB SPL).

In addition, there is the serious problem of parasitic vibration-induced signals (in addition to the desired signals directly caused by sound pressure). If the microphone were positioned outside the speaker enclosure, these parasitic signals would arise due to the necessity to fix the microphone on an angle arm close to the speaker membrane. Vibrations of the arm may contribute significantly to the microphone output, thus degrading the fidelity of the speaker system.

Another general tendency in the field of subwoofer design is also relevant to this invention. The principle of usage of speaker drivers having resonance frequency lower than the cut-off frequency of the sound-reproducing system (to avoid sound distortions), is used both in systems with and without MFB. The typical rule in the field is that the speaker driver attached to the speaker enclosure should have resonance frequency lower than 20 Hz. Practically, this requirement results in increase of the speaker driver size, complicated (frequently custom) design, and in the necessity to use high activating electrical power, sometimes up to 1 kW or more.

In recent years, gas mass flow sensors based on thermoanemometer-type sensing elements (such as Honeywell's AWM series), have been developed and widely used in industry. Sensors encapsulated in a package with a flow channel measure direct gas flow passing through the channel, which depends on pressure drop across the two terminals of the sensor. Therefore, these sensors can be used to measure differential pressure between two non-coincident spatial locations, but not time variations of absolute pressure at a particular point.

Another thermoanemometer-type sensor with sensing elements open to ambient that is able to measure acoustical flows and sound intensity is described in the article by H -E. de Bree, P. Leussink, T. Korthorst, H. Jansen, T S J. Lammerink, M. Elwenspoek, entitled “The μ-flown: A novel device for measuring acoustic flows”, Sensors and Actuators A (1996), v.54, n.1, pp.552-557, and in U.S. Pat. No. 5,959,217. This sensor measures local gas particle velocity which is proportional to spatial gradient of acoustic pressure, but not directly to time-variations in absolute pressure.

It will be appreciated that there is a need for a low-frequency microphone able to record sound frequencies beginning with about 1 Hz and leading up to several tens or a few hundred hertz.

### SUMMARY OF THE INVENTION

One of the purposes of the present invention is to utilize integrated thermoanemometer-type mass flow sensing elements for measurements of time-variations in absolute pressure, abbreviated as absolute pressure variations (APV), to distinguish typical membrane-type microphones from flow-based microphones, to improve resolution and accuracy of such measurements, especially at low frequencies and improve immunity of the measurements to other inertial mechanical vibrations. The invented APV sensor (or sensors) are proposed further to be used in MFB of a speaker system, and in the creation of a hybrid microphone with superior low-frequency performance.

The objects of the present invention are therefore to:

(1) Utilize integrated thermoanemometer-type mass flow sensing elements for measurements of time-variations in absolute pressure. The absolute pressure variations (APV) sensor is invented so as to provide high sensitivity down to several hertz and below, and up to at least 100 Hz. It is also invented so as to have a small size and be immune to inertial (non-sound-pressure) vibrations;

(2) Utilize motional feedback (MFB) based on an APV sensor to improve the frequency response or sound reproduction and recording systems at low frequencies and reduce sound distortions;

(3) Utilize MFB based on an APV sensor having special design and immune to vibrations to improve the frequency response at low frequencies, reduce sound distortions and provide stable operation under external vibrations/acceleration;

(4) Utilize MFB based on the above mentioned sensor in a sound reproducing system wherein relatively small speaker drivers and speaker enclosures are desired.

(5) Utilize MFB based on the above mentioned sensor in a sound reproducing system wherein speaker drivers with resonance frequency higher than the lowest reproducing frequency are used;

(6) Utilize the APV sensor to create a hybrid microphone with superior low-frequency performance;

(7) Propose a mode of motional feedback, enabled by the APV sensor (or accelerometer), which does not require feeding back to the input of the amplifier, and which allows the speaker and feedback system to remain powered only from the (single-cable) audio signal from the amplifier;

(8) Utilize the APV sensor in such a system of motional feedback without connection back to the input of the amplifier.

According to the invention, there is provided a transducer device for time variations of pressure comprising a chamber, a restrictive flow channel communicating gas from ambient to the chamber across a thermal gas flow sensor, characterized in that the sensor comprises thermoanemometer-type sensing elements integrated on a substrate and having a low thermal inertia. The low thermal inertia may allow the device to have an upper cut-off frequency higher than about 50 Hz, and advantageously about 150 Hz. The channel may be coupled with the sensor such that gas flow velocity over the sensing elements is the same as or greater than in the channel, so as to improve sensitivity. Preferably, the sensor is provided inside the chamber, and the channel may also be provided inside the chamber. The thermoanemometer-type sensing elements are preferably mounted inside the channel and a remainder of the sensor outside the channel.

The device preferably has at least two sensors, the sensors and the channel being arranged geometrically to provide signals which can be combined to cancel an inertial vibration-related component.

The present invention also provides a hybrid microphone comprising a membrane-based microphone sensitive for a normal range of audio signals, a thermoanemometer-based microphone comprising the transducer device according to the invention for detecting low-frequency audio signals, and a combiner circuit for combining an output of the membrane-based microphone and the thermoanemometer-based microphone to provide a combined output signal with good response from low audio frequency to at least normal audio frequency.

The invention also provides a motional feedback (MFB) speaker apparatus comprising a speaker, a circuit for modifying an input audio signal to compensate for low frequency attenuations and distortions introduced by the speaker in response to a feedback signal, and a microphone for generating a feedback signal, characterized in that the microphone comprises a microphone comprising the transducer device or the hybrid microphone according to the invention. The channel is preferably perpendicular to an axis of sound propagation of the speaker.

The invention further provides a motional feedback (MFB) speaker apparatus comprising a speaker, a circuit for modifying an input audio signal to compensate for low frequency attenuations and distortions introduced by the speaker in response to a feedback signal, and a microphone for generating a feedback signal, characterized in that the circuit applies a variable attenuation to the audio signal between an amplifier source and the speaker, the variable attenuation having a base level which is modulated to provide the compensation.

Preferably, the circuit applies a variable attenuation to the audio signal between an amplifier source and the speaker, the variable attenuation having a base level which is modulated to provide the compensation. The circuit may be powered by the audio signal. Preferably, the circuit is housed in a housing adapted to be positioned next to the speaker, the housing comprising a mounting for holding the microphone in front of the speaker. The housing can also provide a base or stand for the speaker.

The variable attenuation is preferably provided by a pulse width modulation (PWM) circuit operating at a high frequency which does not cause audible interference in the speaker. The circuit may also separate a low frequency component of the audio signal from a medium/high frequency component, modifies only the low frequency component for the compensation, demodulate the compensated low frequency component, and mix the compensated demodulated low frequency component with the medium/high frequency component.

### BRIEF DESCRIPTION OF THE DRAWINGS

The invention will be better understood by way of the following detailed description of a preferred embodiment and other embodiments with reference to the appended drawings, in which:

FIG. 1 shows a schematic drawing of the APV sensor having one thermoanemometer-type sensing element;

FIG. 2 shows a schematic drawing of the sound reproducing system containing absolute pressure variations sensor based on flow-sensitive element in motional feedback;

FIG. 3a is an oblique view of a flow channel having an integrated thermoanemometer-type sensing device fit into a recess cut into the flow channel tube;

FIG. 3b is a sectional side view of the flow channel having an integrated thermoanemometer-type sensing device fit into a recess cut into the flow channel tube, as shown in FIG. 3a;

FIG. 4 shows schematically the APV sensor having two thermoanemometer-type sensing elements arranged along one flow channel;

FIG. 5 shows schematically the APV sensor placed inside the chamber;

FIG. 6 shows the experimentally-determined frequency response of the APV sensor in configuration with a replaceable chamber;

FIG. 7 schematically illustrates the APV sensor with thermoanemometer-type sensing element attached to the speaker;

FIG. 8 schematically shows the APV sensor immune to vibration, consisting of two thermoanemometer-type flow-sensitive elements;

FIG. 9 shows the speaker system with an APV sensor placed inside a speaker enclosure;

FIG. 10 shows an example of experimentally measured frequency response of a speaker system at low frequencies without and with MFB;

FIGS. 11a,b,c,d show experimentally the measured frequency spectrum of sound generated by the speaker system excited by a 25 Hz tone without and with MFB, respectively;

FIG. 12 is a schematic diagram of a simple hybrid microphone comprising the APV sensor and a capacitive-membrane-type microphone in combination;

FIGS. 13a and 13b are graphs of response vs. time of the hybrid microphone and an instrumentation microphone, respectively, with acoustic inputs sealed, to a common inertial vibratory disturbance at roughly 5 Hz;

FIGS. 14a and 14b are graphs of response vs. time of the hybrid microphone and an instrumentation microphone, respectively, with acoustic inputs open, to a common movement, similar to the movement in FIG. 13;

FIG. 15a is a schematic diagram of a circuit providing MFB with variable attenuation to the speaker signal by means of pulse width modulation using a PWM frequency above audible frequencies, in which power for the circuit is derived from the audio speaker signal itself; and

FIG. 15b is a schematic diagram of a physical set-up of the MFB system according to the embodiment of FIG. 15a in which the circuit is provided in a base on which the speaker sits, the base supporting the microphone mount.

### DETAILED DESCRIPTION OF THE PREFERRED EMBODIMENT AND OTHER EMBODIMENTS

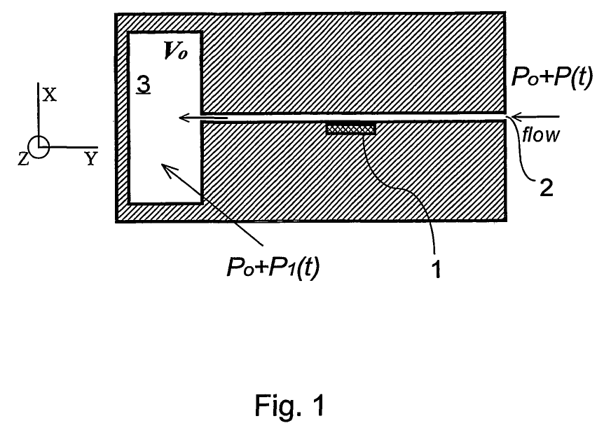

In order to utilize an integrated mass flow sensor based on thermoanemometer-type sensing elements for measuring of absolute pressure variations, a special pneumatic housing of the sensor is used. In general, the sensor according to the preferred embodiment contains at least one thermoanemometer-type sensing element 1 placed in the container housing the flow channel 2. The channel is connected to the closed chamber 3 with rigid walls and volume Vₒ as shown in FIG. 1. The other end of the channel is open to the ambient. The operating principle of the sensor is explained as follows.

Let absolute pressure of ambient gas be expressed as Pₒ+Pₐ(t), where the term Pₐ(t) (|Pₐ(t)|<<Pₒ) represents pressure variations with respect to a constant Pₒ. These variations, Pₐ(t), are the signal of interest, the absolute pressure variations (APV).

The pressure inside the closed chamber 3 varies as Pₒ+P_{c}(t), where P_{c}(t) is in general different from Pₐ(t). Under these conditions, the pressure differential applied to the channel 2 causes gas flow through the channel 2, F(t), which can be found from the equation

$$RF ( t ) + L \frac{ⅆ F ( t )}{ⅆ t} = {P}_{a} ( t ) - {P}_{c} ( t ) ,$$

where:

$$R = \frac{128 η l}{{D}^{4}}$$

is the flow impedance (flow resistance) of the channel 2, D and I are the inner diameter and the length of the channel 2, η is air viscosity;

$$L = \frac{4 n ρ l}{π {D}^{2}}$$

is fluid inductance of the channel 2, ρ is air density, n is 1 if the velocity profile is blunt and n=4/3 if parabolic.

It can be shown that at low frequencies, when contribution of the flow channel inductance can be neglected, the flow caused by the absolute pressure variations can be found from the equation

$$\frac{ⅆ F ( t )}{ⅆ t} + \frac{F ( t )}{τ} = \frac{1}{R} \frac{ⅆ {P}_{a} ( t )}{ⅆ t} ,$$

where

$$τ = \frac{{V}_{o} R}{{P}_{o}} .$$

In the case of sinusoidal pressure variations, Pₐ(t)=ΔP sin(2πft), where ΔP and f are pressure amplitude and frequency respectively, gas flow passing through the channel equals

$$\begin{matrix}F ( t ) = {F}_{o} sin ( 2 π f t - ϕ ) \\ {F}_{o} = Δ {P ( 1 + \frac{1}{{( 2 π f τ )}^{2}} )}^{- 1 / 2} \\ ϕ = arctan \frac{1}{2 π τ}\end{matrix}$$

The flow sensor therefore has low cut-off frequency

$${f}_{L} = \frac{1}{2 π τ} = \frac{{P}_{o}}{2 π {V}_{o} R} .$$

At higher frequencies, sensitivity of the sensor is limited by inductance of the flow channel 2 and high cut-off frequency is defined as

$${f}_{H} = \frac{R}{2 π L} = \frac{192 η}{ρ {D}^{2}} .$$

a sensor with the flow channel 2 with inner diameter of D=2 mm, f_{H}660 Hz (f_{H}=295 Hz at D=3 mm).

Further, the sensitivity of the sensor is additionally limited at higher frequencies due to the inertia of the thermoanemometer-type sensing elements 1. Typically, frequency response of modern thermoanemometers extends up to 150-200 Hz, but can be improved up to several hundred Hz. This pressure-sensitivity depends principally on the careful design of the flow-sensitive elements within the flow channel. In particular, it is desirable to accumulate the entire flow in the vicinity of the heated elements. While the mass flow in the channel is constant, it may be desirable for greater sensitivity to cause the flow to increase in velocity near the sensing elements, as for example by using a constriction.

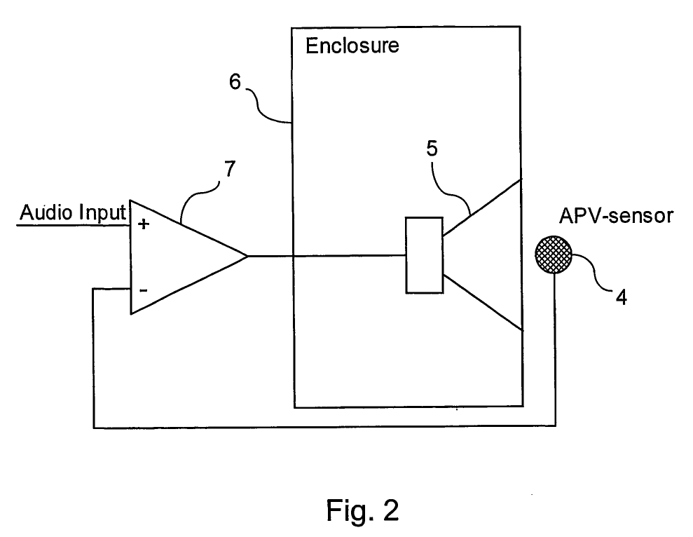

Referring to FIG. 2, the sound reproducing system comprises the speaker driver 5 attached to the speaker enclosure 6. The input audio signal is applied to the power amplifier 7 which drives the speaker driver 5. The absolute pressure variations (APV) sensor 4 with flow sensitive-element 1 is located near the speaker driver 5 outside the speaker enclosure 6 and detects generated sound pressure. Its output signal is amplified and filtered (if necessary) by the electronic processing circuitry. The processed signal is fed to the inverting input of the power amplifier 7. This negative feedback assures that the generated sound pressure faithfully tracks the audio signal.

In FIG. 2, the MFB scheme shows the output from the APV sensor 4 being fed back to the input of the amplifier 7. In practice, this imposes certain restrictions/conditions on the amplifier/speaker system. In the case where the amplifier 7 is not integrated inside the speaker enclosure 6, an additional cable from the speaker to the amplifier is needed. Also, the amplifier 7 must offer access to its input, which is not necessarily the case.

In response to these inconveniences, this invention provides a motional feedback scheme which can be used in a standalone speaker, operable from only a single output line from the amplifier, not requiring any signal to be fed back to the amplifier input. There are several possible embodiments of this scheme.

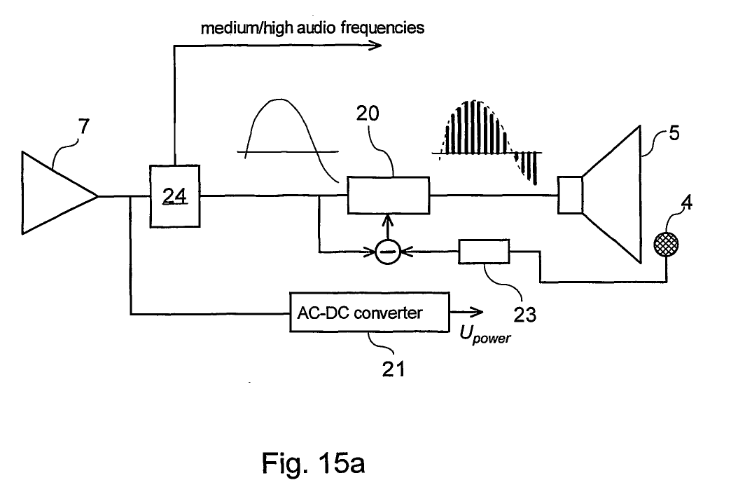

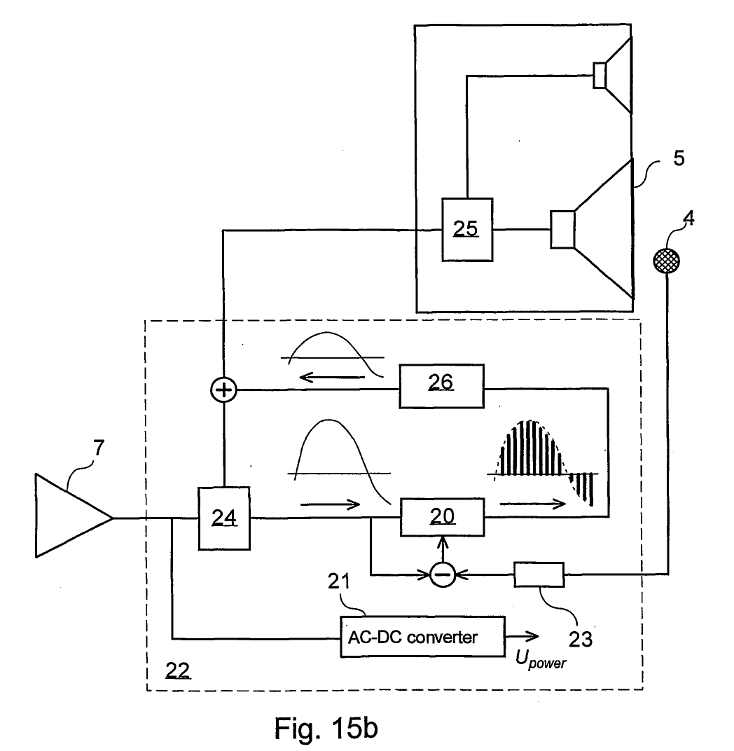

One embodiment is shown in FIGS. 15a and 15b, where the audio signal is separated by a cross-over filter 24 into low and medium/high frequency components. The medium/high component is suitably attenuated by the filter 24 or another circuit element and may be fed directly to a medium/high speaker driver (not shown) and possibly to the speaker driver 5 in the embodiment of FIG. 15a. The low frequency component is fed to the speaker driver 5 through an electronic module 20 which provides pulse-width modulation of the audio signal. The signal from the APV sensor 4, corresponding to the measured sound pressure, is amplified by module 23, and compared with the input low frequency electrical signal. Depending on the result of this comparison, the electronic module 20 changes the parameters of the pulse-width modulation to compensate for distortions. It is important that the normal level of pulse-width modulation not be at maximum, so that the delivered power can be both increased or decreased if necessary to compensate for distortions.

Typically, such circuit elements require electric power at the level of approximately tens of mW, while the power delivered to the speaker driver from the amplifier 7, may reach hundreds of watts, which makes it possible to power the whole electronic scheme from the audio signal line, by the addition of an AC-DC converter 21, to power the electronics, without significantly degrading the input signal. When the input power is low or zero, distortions are least likely, and the compensation circuit need not operate.

The invention also includes the possibility of creating an independent housing 22, as shown in FIG. 15b to be used with a regular speaker, which accepts the input from the amplifier 7, senses the sound pressure out in front of the speaker, implements the compensation, and applies the correct signal to the speaker input, as a retro-fit unit. To provide a stand-alone unit able to be used with a standard speaker having its own cross-over filter 25, the unit 22 has a demodulator unit 26 to remove the high frequency components from the PWM low frequency component signal, prior to recombining the low and medium/high components for output to the speaker.

The embodiments of FIGS. 15a and 15b are but some examples. In general, powering the circuitry using the audio signal is highly desirable, but not always necessary. Likewise, the use of PWM as a variable attenuator is desirable but not always necessary. This embodiment may allow an essentially stand-alone add-on to a conventional low-end speaker to provide audiophile results. Since the attenuation would require that the power from the amplifier 7 be increased during normal listening, the device 22 should be provided to all speakers. A by-pass or off switch is a useful accessory, as is an adjustment capability for the base level attenuation. An indicator for indicating when the MFB is effective is also desirable, as is an indicator for indicating when the MFB is failing to provide full compensation.

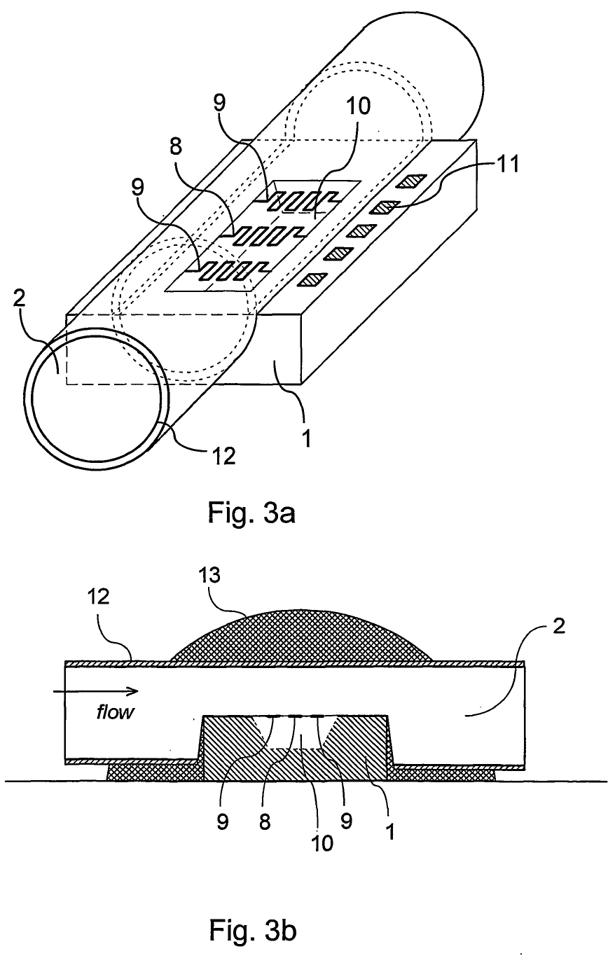

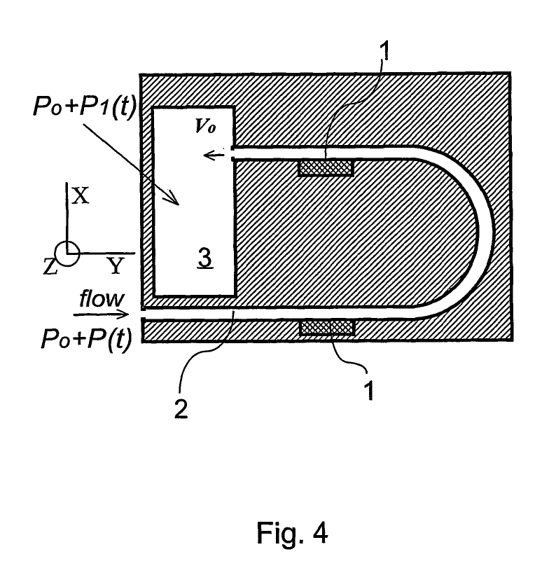

FIGS. 3a and b show the most important functional parts of the APV sensor according to the preferred embodiment, namely where the thermoanemometer is attached to the flow channel. In this embodiment, the capillary tube 12 has a cut-out slot, into which the silicon substrate 1 is fitted. The capillary tube 12 is attached such that it entirely and symmetrically covers the integrated heating and sensing elements. In this particular embodiment, the micromachined central heater 8, and micromachined temperature-sensing thermoresistors 9, are suspended over the cavity 10 in the silicon substrate 1, which is of any suitable shape and provides high sensitivity and low thermal inertia for the device. The size of the thermoresistors may be as low as 100 μm, therefore the inner diameter of the flow channel 2 may have approximately the same size (100-200 μm), which allows the entire gas flow to be accumulated in a small cross section, which gives high gas velocity and high sensitivity of the sensor. The use of a capillary tube 12 with small inner diameter, allows one to significantly increase the flow impedance, and reduce the required volume of the attached chamber, which is extremely important for miniaturization of the entire sensor. FIG. 3b shows schematically a cross-section of the sensor, wherein epoxy 13 or other suitable adhesive is used to attach and seal the tube 12 to the silicon substrate 1. The tube 12 needs to cover only the integrated thermoanemometer sensing elements 8,9. For example the wire-bonding pads 11, for electrical connections, need not be within the flow channel 2.

FIG. 1 schematically illustrates the absolute pressure variation sensor consisting of the flow channel 2 connected to the closed chamber and the thermoanemometer-type sensing element 1. Proper design of the pneumatic part of the sensor (channel 2 and chamber 3) allows one to establish frequency f_{L} at the level required for a particular application. For example, a sensor having flow impedance of the channel 2, R=1000 Pa.s/ml, has f_{L}=15 Hz, when it is connected to a chamber 3 with volume Vₒ=1 ml. To lower the cut-off frequency, say to 0.3 Hz, a chamber 3 with greater volume, 50 ml, should be used.

Regulation of the cut-off frequency, f_{L}, can be also accomplished by proper design of the flow channel 2. For example, an additional capillary tube with length of 1 cm and inner diameter of 0.2 mm connected in series to the sensor flow channel 2 increases total flow impedance by approximately 14000 Pa.s/ml and lowers the cut-off frequency of the sensor. In this case, we need only 0.3 ml to reach 3 Hz instead of 100's of ml.

Immunity to vibrations of the APV sensors according to the preferred embodiment is explained by the following physical phenomena. The sensor shown schematically in FIG. 1 has sensitivity to vibrations acting in the direction parallel to the Y axis. Vibration sensitivity is caused by vibration-induced shift of the heated volume of gas, in the vicinity of the heater of the thermoanemometer-type sensing element 1. An important feature of the thermoanemometer is that it has vibration sensitivity in the same direction as flow sensitivity. The sensor has negligible sensitivity to vibrations acting in directions parallel to the XZ plane because no temperature differential across the sensing element is produced in this case. Therefore the invented sensor can be used to measure absolute pressure variations under vibrations acting in one preferred direction or in one preferred plane. Immunity to vibrations in this case is accomplished by orienting the sensor in such a way that vibrations are perpendicular to the flow channel 2 in the vicinity of the thermoanemometer-type sensing element 1.

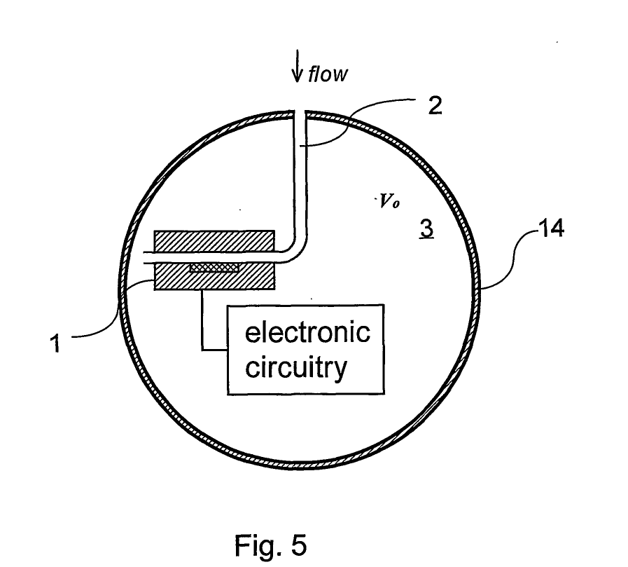

To provide immunity to vibrations acting in all directions, the sensor schematically shown in FIG. 4 is provided. It contains two thermoanemometer-type sensing elements 1 arranged along one flow channel 2 connected to the closed chamber 3. Immunity to vibrations acting in the direction parallel to the XZ plane is explained by the same reasons as for one-element sensor. Immunity to vibrations acting in the Y-direction is explained by the following reasons. The gas flowing through the channel 2 passes through the thermoanemometers 1 in opposite directions. Therefore heated volumes of gas near the heaters in both thermoanemometers are also shifted in opposite directions causing inverted output signals. When acceleration is applied in the direction parallel to gas flow (and axis Y), heated volumes of gas are shifted in the same direction for both thermoanemometers causing increments in output signals which are the same for both sensors. The output signals of the two flow-sensitive elements 1 are then processed by electronic circuitry, such that one signal is subtracted from the other.

Sensor-output-1=(flow)+(acceleration)

Sensor-output-2=−(flow)+(acceleration)

Sensor-output-1−Sensor-output-2=2*(flow)

If the two thermoanemometers 1 are identical, sensitivity to acceleration of the whole APV sensor can be reduced theoretically to zero. In practice, the immunity to acceleration may be limited by mismatch of the two thermoanemometers and calibration of their sensitivities. Reference is had to Applicant's copending PCT publication WO01/18496 published on Mar. 15, 2001 for greater detail on the flow sensor construction and design.

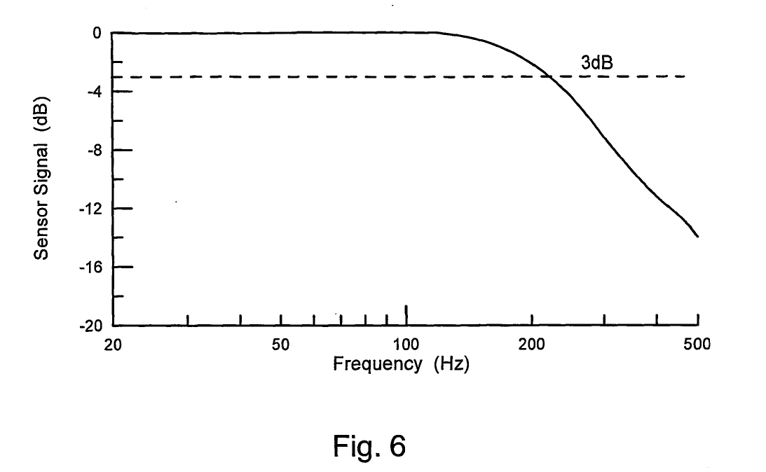

FIG. 5 illustrates the APV sensor containing a thermoanemometer-type sensing element 1 located inside the chamber 3. This design provides-better mechanical protection of the sensing element by its additional enclosure 14. The disclosed construction is preferable when a chamber 3 is designed having volume Vₒ of several ml and higher. The walls of the chamber can be also used to enclose associated sensor signal processing circuitry.

The following should be noted:

(A) APV sensors additional to those shown in FIGS. 1, 3-5 can be proposed as a combination of different flow assemblies and volume chambers. For example, two-element sensing element (shown on FIG. 3) can be placed inside the chamber 3 as shown on FIG. 5. The particular design should be chosen so to optimally accommodate the application conditions.

(B) The particular construction of the thermoanemometer-type sensing element 1 is not important for the disclosed invention except that it should have the following features. It must have bi-directional sensitivity to gas flow passing through the channel 2 and may contain two temperature-sensitive elements, and one electric heater located between the temperature-sensitive elements. It also may contain only two self-heated temperature-sensitive elements, see the mentioned article by H -E. de Bree et al. in Sensors and Actuators A (1996), v.54, n.1, pp.552-557. The particular design of its functional elements, which may be thermoresistors, thermopiles or other means transforming temperature into an electric signal, is also not important for the disclosed invention.

An experimental prototype was manufactured and tested to prove the APV sensor (see FIGS. 3a and 3b). The prototype is based on the micromachined thermoanemometer 1 according to FIGS. 3a and 3b attached to the flow channel 2 having flow impedance of approximately 500 Pa.s/ml. The sensing element of the thermoanemometer 1 contains three polysilicon resistors thermally isolated from silicon substrate and the package. The central resistor 8 operates as the heater while two others 9, located symmetrically on both sides of it, detect temperature differential caused by gas flowing through the channel 2. The flow channel 2 is connected to the replaceable chamber with rigid walls.

The time response τ the sensor connected to a chamber with volume of 10 ml was measured by applying a step overpressure to the sensor input. After the signal reaches its maximum, several milliseconds after the beginning of the step, it reduces exponentially due to equalization of pressure inside the chamber and at the input of the sensor. The time constant of this equalization was measured to be τ=0.05 s from which one calculates the cut-off frequency f_{L}=3 Hz.

The same experiment was repeated with a chamber volume of 60 ml. The measured time constant was τ=0.3 s corresponding to f_{L}=0.5 Hz. The obtained data are in agreement with theoretical estimates which predict that the cut-off frequency will decrease with increasing of volume Vₒ.

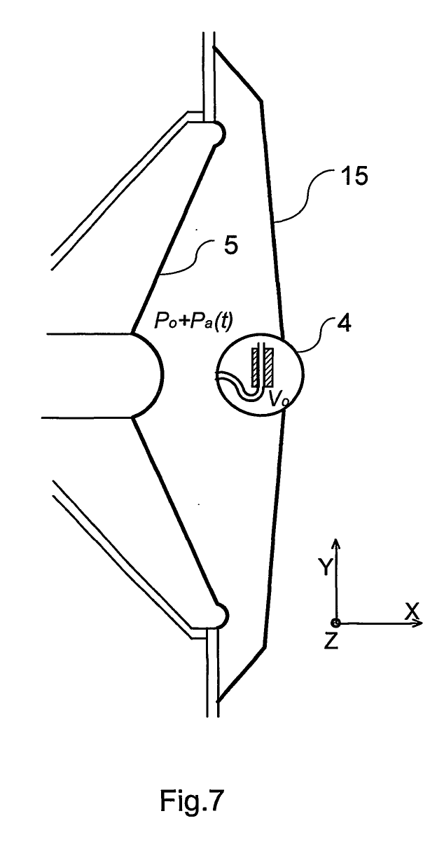

Frequency response of the APV sensor is shown in FIG. 6. Pressure variations (sound pressure) were generated by an audio speaker driver and controlled by an instrumentation microphone (AUDIX TR40) having flat (±1 dB) frequency response from 20 Hz to 20 kHz. The measured frequency response is demonstrated to be flat (within 3 dB) approximately up to 220 Hz. This same high cut-off frequency of f_{H}=220 Hz was found for both chambers volumes of 10 ml and 60 ml.

### Speaker System with Motional Feedback Utilizing APV Sensor

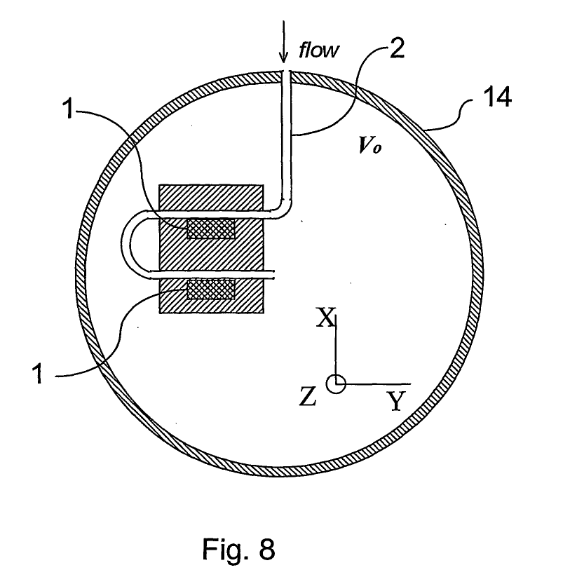

FIG. 7 illustrates schematically the APV sensor 4 with thermoanemometer-type sensing element 1 (as flow-sensitive element) attached to the speaker enclosure with a supporting arms 15. The sensor contains flow-sensitive element assembled in a package with the flow channel. The flow channel allows air flow between the ambient and the closed chamber (inner volume Vₒ of the sensor enclosure). The sound pressure generated by the speaker membrane results in pressure drop across the flow channel (from its input to output) and hence gas flow. In the vicinity of the flow-sensitive element, gas flows in the direction parallel to the X axis.

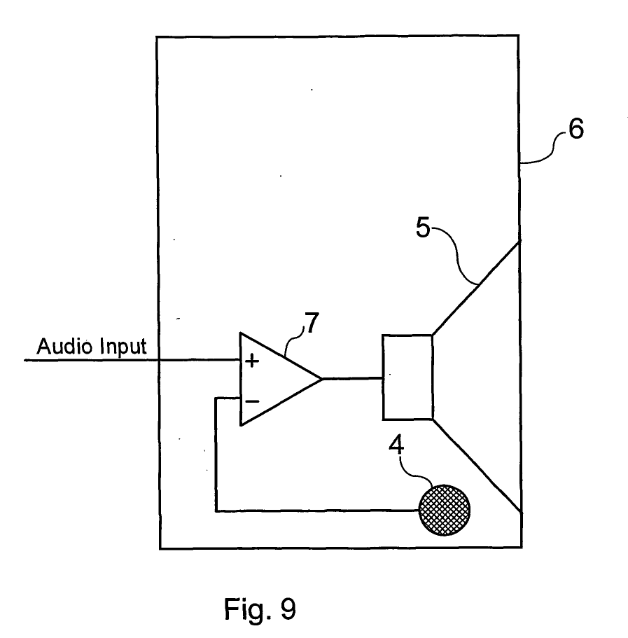

The sensor shown in FIG. 7 has negligible sensitivity to vibrations acting in directions parallel to the XZ plane as was explained above. The sensor shown in FIG. 8, with better immunity to vibrations, can be also used in MFB.

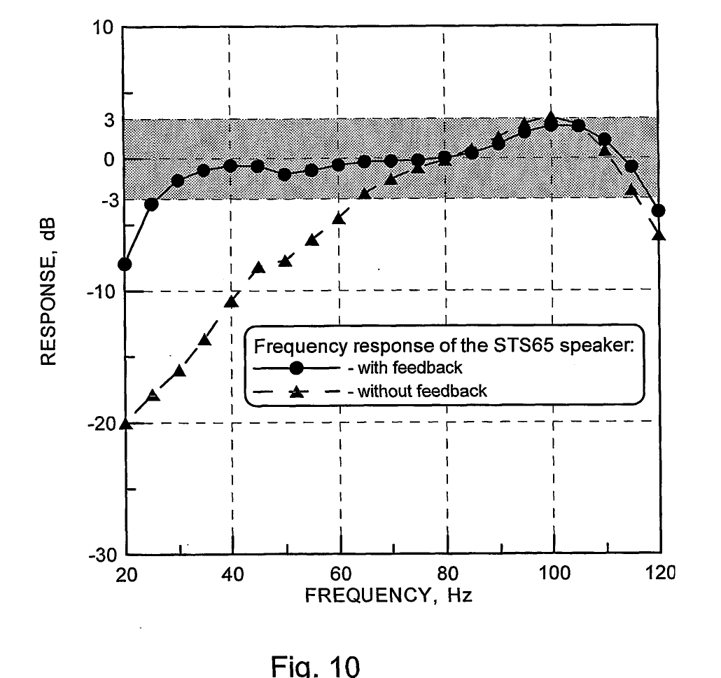

FIG. 9 shows another possible embodiment of a speaker system with APV sensor 4 placed inside the speaker enclosure 6. It is well known that at low frequencies, acoustic pressure inside the sealed enclosure is proportional to the displacement of the speaker cone while acoustic pressure outside the enclosure is proportional to the cone acceleration. It should be noted that accomplishment of MFB is possible in both cases, with the APV sensor 4 placed inside or outside of the enclosure 6. For the case of an APV sensor 4 placed inside the enclosure 6, both the signal processing (frequency filtering) should be changed, and the sensor itself should be adapted to accommodate much higher acoustic pressure up to 180-200 dB SPL. The invented structure of the APV sensor 4 allows easy adaptation to this range. An additional capillary tube with small inner diameter connected in series to the sensor restricts gas flow through the thermoanemometer 1 and the shifts operating range of the device to higher pressures.

To prove the effectiveness of the disclosed concept of the MFB with APV sensor, the following prototyping speaker system and testing procedure were used.

An Optimus™ 2-way bookshelf-sized speaker STS65 was chosen and adapted for the experiments. Having a volume of 16 liters, the speaker contains a 6.5″ speaker driver and a tweeter. Its own crossover filter and the tweeter were disconnected, and an eight-order low-pass filter with cut-off frequency of 120 Hz was installed to restrict the reproducible frequency range to that which is typical for subwoofers.

The APV sensor 4 was attached to the speaker enclosure 6 by three supporting arms 15 at a distance of 1 cm from the speaker membrane as shown in FIG. 7. A thermoanemometer-type sensing element 1 was packaged inside a hollow spherical enclosure with diameter of 3.5 cm (inner volume of approximately 8 ml). The flow channel 2 of the thermoanemometer having pneumatic impedance of approximately 500 Pa.s/ml allows air flow between the ambient and the sealed volume of air inside the spherical enclosure. Gas flow caused by sound pressure at the input of the flow channel is detected by the thermoanemometer 1, and its amplified signal is fed to the inverting input of a 100 watts power amplifier 7. The frequency response of the sensor is flat in the range of 10-220 Hz (within 3 dB).

A testing microphone AUDIX TR40 having flat (±1 dB) frequency response from 20 Hz to 20 kHz was placed at a distance of 1 cm from the speaker membrane to measure generated sound pressure (near-field measurements). Its amplified signal was digitized with 12-bit acquisition board and then visualized, processed and stored by the computer.

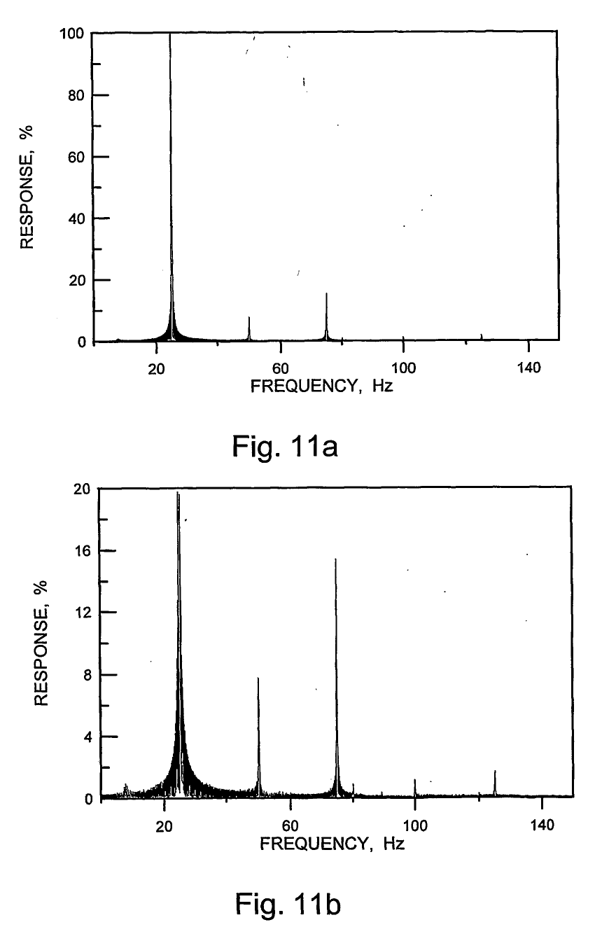

FIG. 10 shows frequency response of the subwoofer without and with feedback. Usage of the invented MFB based on absolute pressure variations sensor allows the extension of the operating frequency range and lower cut-off frequency (at level 3 dB) from 65 Hz to 25 Hz.

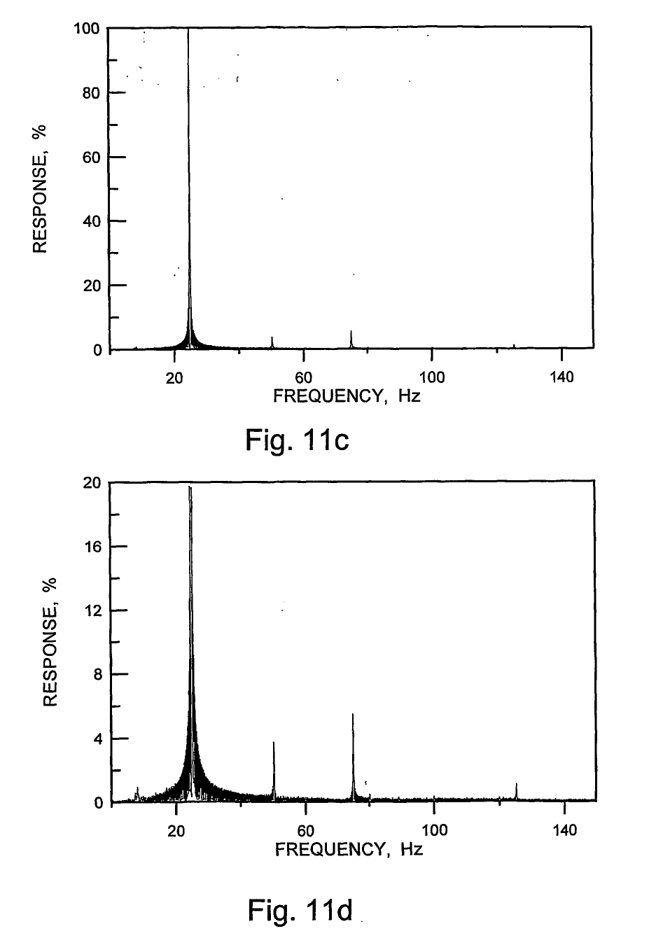

The MFB also provides effective suppression of harmonic distortions of the subwoofer. FIG. 11 shows the frequency spectrum of sound generated by the subwoofer at the same sound pressure level without (a, b) and with (c, d) MFB. Harmonic distortions of the prototyping speaker system were tested in a frequency range from 20 Hz to 110 Hz. FIGS. 11a and b show the data without MFB on two different vertical scales, while FIGS. 11c and d show the data with MFB on the same two vertical scales. Evident and effective suppression of higher-order harmonics provided by the MFB according to the invention was experimentally confirmed in the whole operating frequency range. No unwanted additional distortions due to the natural resonance of the speaker system existing at approximately 50 Hz (which is higher than the lower operating frequency of 20 Hz) were found.

The embodiment described above is not the only possible embodiment of the use of the APV sensor in a sound system with MFB. Ported (vented) speaker systems operate in a more complex manner, since there are essentially two sources of sound (the speaker driver and the port). In principle, feedback systems consisting of one or several APV sensors can be constructed, with sensors positioned at various locations inside and/or outside of the speaker enclosure.

### Hybrid Microphone Utilizing APV Sensor

Typical membrane-type microphones have certain performance limitations. The response at low frequencies is often limited by an inherent increase in intrinsic (self) noise, and such microphones are typically responsive to parasitic mechanical vibrations as well as the signals (sound pressure variations) of interest. The invented APV sensor has better signal-to-noise-ratio (SNR) at low frequencies and high immunity to vibrations. However, its high-cut-off frequency is limited to be in the range of several hundred Hz, which is not enough to cover the whole range of operation of typical microphones. In order to take advantage of the characteristics of both the invented APV sensor and typical inexpensive microphones, they can be used in combination to produce a hybrid microphone, replacing the poor low-frequency performance of the inexpensive membrane-type microphone with the excellent low-frequency performance of the APV sensor. This combination will have the enhanced SNR at low frequencies, as well as reduced sensitivity to mechanical vibrations at low frequencies.

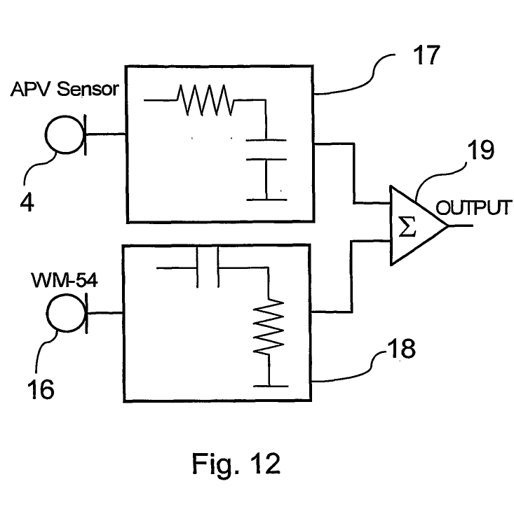

FIG. 12 shows a schematic electrical diagram of how the two components would be connected in a hybrid microphone, to obtain a flat frequency response over the whole range (including cross-over). An experimental prototype, schematically presented in FIG. 12, consists of an APV sensor 4, described above, and a commercially-available Panasonic WM-54 microphone 16, along with a first-order low-pass electrical filter 17, and a first-order high-pass electrical filter 18. The two filtered signals from the sensors 4 and 16 are summed using summing amplifier 19.

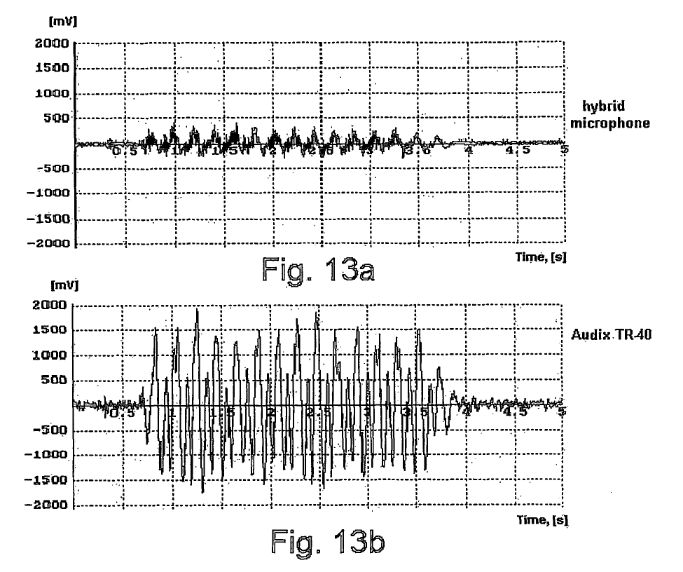

Experiments have been done to compare the performance of an experimental prototype hybrid microphone shown in FIG. 12, with an instrumentation microphone Audix TR40, having flat frequency response±1 dB from 20 Hz to 20 kHz. These two microphones were placed on a common rigid mechanical support. The gain of the two instruments was adjusted such that their output electrical signals were of equal amplitude for a given 30 Hz acoustic input of 94 dB. Then the acoustic inputs were sealed, such that the instruments were isolated from acoustic disturbances, in order to highlight sensitivity to vibrations at low frequency (such as roughly 5 Hz), and they were subjected to mechanical motions. FIG. 13 shows the reactions of the two instruments, demonstrating that the hybrid is much more immune to these (common) mechanical disturbances. Then, the acoustic inputs of the instruments were re-opened, and similar mechanical motions were applied. With acoustic inputs open, the instruments can measure these low-frequency pressure variations induced by the motion.

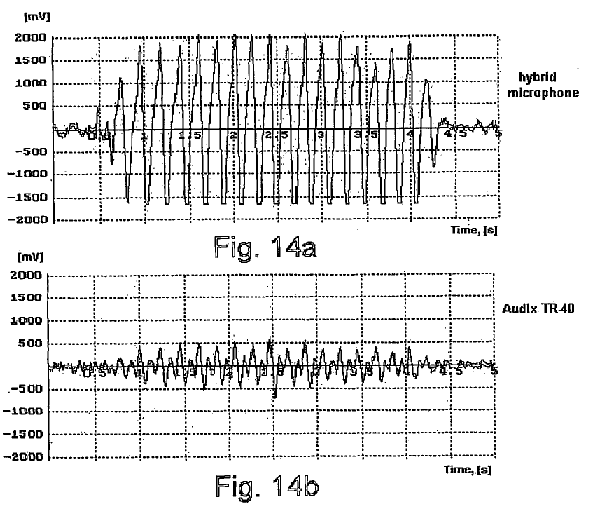

FIG. 14 shows the reactions of the instruments. The hybrid demonstrates high sensitivity to these pressure variations (note that the scales of the two figures are different), while the TR40 response is much lower due to suppression of frequencies lower than 20 Hz by a high-order high-pass filter included in the microphone. Of course, part of the low-frequency range can be filtered, depending on the desired application, such as 10-20 Hz for music, for example.

It is known that capacitive (electret) membrane-type sound-pressure transducers are often highly-resistive signal sources. One of the features of modern microphones is that they are highly-resistive signal sources. It is well-known from general physics and electronics, that if one works with highly-resistive sources, there is the problem of avoiding electromagnetic interference (EMI), which can be solved by proper grounding, shielding, etc. Fortunately (for this invention), thermo-anemometer-type sensors have low resistance from hundreds of ohms to several kΩ. As a result, they are much less susceptible to EMI.

1. A transducer device for time variations of pressure comprising a chamber, a restrictive flow channel communicating gas from ambient to said chamber across a thermal gas flow sensor,
characterized in that
said sensor comprises thermoanemometer-type sensing elements integrated on a substrate and having a low thermal inertia.

2. The device as claimed in claim 1, wherein said low thermal inertia allows said device to have an upper cut-off frequency higher than about 50 Hz.

3. The device as claimed in claim 1, wherein said low thermal inertia allows said device to have an upper cut-off frequency higher than about 100 Hz.

4. The device as claimed in claim 1, wherein said low thermal inertia allows said device to have an upper cut-off frequency higher than about 150 Hz.

5. The device as claimed in any one of claims 1 to 4, wherein said channel is coupled with said sensor such that gas flow velocity over said sensing elements is the same as or greater than in said channel.

6. The device as claimed in any one of claims 1 to 5, wherein said sensor is provided inside said chamber.

7. The device as claimed in claim 6, wherein said channel is also provided inside said chamber.

8. The device as claimed in any one of claims 1 to 7, wherein said thermoanemometer-type sensing elements are inside said channel and a remainder of said sensor being outside said channel.

9. The device as claimed in any one of claims 1 to 8, comprising at least two said sensors, said sensors and said channel being arranged geometrically to provide signals which can be combined to cancel an inertial vibration-related component.

10. A hybrid microphone comprising a membrane-based microphone sensitive for a normal range of audio signals, a thermoanemometer-based microphone comprising a transducer device as defined in any one of claims 1 to 9 for detecting low-frequency audio signals, and a combiner circuit for combining an output of said membrane-based microphone and said thermoanemometer-based microphone to provide a combined output signal with good response from low audio frequency to at least normal audio frequency.

11. A motional feedback (MFB) speaker apparatus comprising a speaker, a circuit for modifying an input audio signal to compensate for low frequency attenuations and distortions introduced by said speaker in response to a feedback signal, and a microphone for generating a feedback signal,
characterized in that
said microphone comprises a microphone comprising one of: a transducer device as defined in any one of claims 1 to 9; and a hybrid microphone as defined in claim 10.

12. The speaker apparatus as claimed in claim 11, wherein said circuit applies a variable attenuation to said audio signal between an amplifier source and said speaker, said variable attenuation having a base level which is modulated to provide said compensation.

13. The speaker apparatus as claimed in claim 11 or 12, wherein said channel is perpendicular to an axis of sound propagation of said speaker.

14. A motional feedback (MFB) speaker apparatus comprising a speaker, a circuit for modifying an input audio signal to compensate for low frequency attenuations and distortions introduced by said speaker in response to a feedback signal, and a microphone for generating a feedback signal,
characterized in that
said circuit applies a variable attenuation to said audio signal between an amplifier source and said speaker, said variable attenuation having a base level which is modulated to provide said compensation.

15. The speaker apparatus as claimed in any one of claims 11 to 14, wherein said circuit is powered by said audio signal.

16. The speaker apparatus as claimed in any one of claims 11 to 15, wherein said circuit is housed in a housing adapted to be positioned next to said speaker, said housing comprising a mounting for holding said microphone in front of said speaker.

17. The speaker apparatus as claimed in claim 16, wherein said housing provides a base or stand for said speaker.

18. The speaker apparatus as claimed in any one of claims 12 to 17, wherein said variable attenuation is provided by a pulse width modulation (PWM) circuit operating at a high frequency which does not cause audible interference in said speaker.

19. The speaker apparatus as claimed in claim 18, wherein said circuit separates a low frequency component of said audio signal from a medium/high frequency component, modifies only said low frequency component for said compensation, demodulates said compensated low frequency component, and mixes said compensated demodulated low frequency component with said medium/high frequency component.

20. The speaker apparatus as claimed in any one of claims 12 to 17, wherein said circuit separates a low frequency component of said audio signal from a medium/high frequency component, modifies only said low frequency component for said compensation, and mixes said compensated low frequency component with said medium/high frequency component.
